(19) United States
(12) Patent Application Publication
(10) Pub. No.: US 2018/0238859 A1
(43) Pub. Date: Aug. 23, 2018

(54) PREDICTIVE METHOD FOR CHARACTERIZING THE SENSITIVITY OF A TUMOUR IN RESPONSE TO A DNA-BREAKING TREATMENT

(71) Applicants: NEOLYS DIAGNOSTICS, Lyon (FR); INSTITUT NATIONAL DE LA SANTÉ ET DE LA RECHERCHE MÉDICALE, Paris (FR); CENTRE LEON BERARD, Lyon (FR); CENTRE NATIONAL DE LA RECHERCHE SCIENTIFIQUE, Paris Cedex 16 (FR); UNIVERSITE CLAUDE BERNARD LYON 1, Villeurbanne Cedex (FR)

(72) Inventors: Nicolas FORAY, Meyrie (FR); Larry BODGI, Lyon (FR); Sandrine PEREIRA, Meyrargues (FR)

(73) Assignees: NEOLYS DIAGNOSTICS, Lyon (FR); INSTITUT NATIONAL DE LA SANTÉ ET DE LA RECHERCHE MÉDICALE, Paris (FR); CENTRE LEON BERARD, Lyon (FR); CENTRE NATIONAL DE LA RECHERCHE SCIENTIFIQUE, Paris Cedex 16 (FR); UNIVERSITE CLAUDE BERNARD LYON 1, Villeurbanne Cedex (FR)

(21) Appl. No.: 15/751,729

(22) PCT Filed: Aug. 16, 2016
(86) PCT No.: PCT/FR2016/052082

§ 371 Date: Feb. 9, 2018

(30) Foreign Application Priority Data
Aug. 19, 2015 (FR) 1501752
Oct. 20, 2015 (FR) 1559961

(51) Int. Cl. G01N 33/50; G01N 33/68

(52) U.S. Cl. CPC G01N 33/5014; G01N 33/5044; G01N 2800/56; G01N 33/68; G01N 2800/52; G01N 33/5011

(57) ABSTRACT

A predictive method of cellular, tissue and clinical radiosensitivity, which is based on the determination and cross-checking of a plurality of cellular and enzymatic parameters and criteria applied to a tumor response. The predictive method characterizes the sensitivity of a tumor in response to a DNA-breaking radiotherapy treatment.

### CROSS-REFERENCE TO RELATED APPLICATIONS

The present application is a National Stage Application of PCT International Application No. PCT/FR2016/052082 (filed on Aug. 16, 2016), under 35 U.S.C. § 371, which claims priority to French Patent Application Nos. 1501752 (filed on Aug. 19, 2015) and 1559961 (filed on Oct. 20, 2015), which are each hereby incorporated by reference in their respective entireties.

### TECHNICAL FIELD

The invention relates to the field of medical radiotherapy, and more particularly the field of radiotherapeutic laboratory methods. The invention relates to a novel predictive method of cellular, tissue and clinical radiosensitivity, which is based on the determination and cross-checking of a plurality of cellular and enzymatic parameters and criteria applied to the tumor response. More particularly, the invention relates to a predictive method for characterizing the sensitivity of a tumor in response to a DNA-breaking radiotherapy treatment.

### BACKGROUND

Non-surgical anti-cancer treatments are generally aimed at inducing the cell death of cancer cells: most of these treatments induce DNA breaks, subsequently inducing the apoptosis of these cells. This is particularly true for tumor treatments using ionizing radiation (radiotherapy). However, there are few methods for predicting the response of a tumor to such an antitumor treatment, and the reliability thereof is not sufficient to guide the strategy of an antitumor treatment in clinical practice (for radiotherapy see: “Radiation Biology, A Handbook for Teachers and Students”, IAEA, 2010, p. 107/108).

It is known that the issue of tissue sensitivity to ionizing radiation is inseparable from those of DNA damage repair mechanisms. Indeed, at a cellular level, ionizing radiation may break certain types of chemical bonds generating free radicals (in particular by peroxidation) and other reactive species causing DNA damage. The damage of DNA by endogenous or exogenous attacks (such as ionizing radiation and free radicals) may result in different types of DNA damage according to the energy deposited in particular: base damage, single-strand breaks and double-strand breaks (DSBs). Non-repaired DSBs are associated with cell death, toxicity and more specifically radiosensitivity. Poorly repaired DSBs are associated with genomic instability, mutagenic phenomena and predisposition to cancer. The body has specific repair systems for each type of DNA damage. In respect of DSB, mammals have two main repair modes: suture repair (strand ligation) and recombination repair (insertion of a homologous or non-homologous strand). This statement is valid regardless of the tissue type, whether normal or tumor tissue.

It is also known that tissue sensitivity to ionizing radiation is very variable from one organ to another and from one individual to another; the idea of “intrinsic radiosensitivity” was conceptualized by Fertil and Malaise (“Inherent cellular radiosensibility as a basic concept for human tumor radiotherapy”, Int. J. Radiation Oncology Biol. Phys. 7, p. 621-629 (1981); “Intrinsic radiosensitivity of human cell lines is correlated with radioresponsiveness of human tumors: Analysis of 101 published survival curves”, Int. J. Radiation Oncology Biol. Phys. 11, p. 1699-1707 (1985)).

As such, the various studies on the therapeutic effects and side-effects of radiotherapy have demonstrated that there are individuals who exhibit particularly high radioresistance, and individuals displaying, on the other hand, radiosensitivity that may range from a clinically recognized but inconsequential side-effect to a lethal effect. Even outside of certain rare cases of extreme radiosensitivity, which appears to be of proven genetic origin, radiosensitivity is thought to stem generally from a genetic predisposition: it is therefore specific to an individual.

Similarly, tumors exhibit a broad spectrum of radiosensitivity which is dependent on both the individual and also on the type of tissue. For example, lymphomas are generally more radiosensitive than sarcomas, as a general rule regardless of the individual's genetic status: for tumors, the “individual factor” may be canceled by the “tissue” factor. In order to assess the benefit/risk ratio of an anticancer treatment, it would therefore be desirable to avail of a predictive test method to be able to determine the minimum cumulative dose that a tumor needs to receive to be sterilized. This question arises firstly in radiotherapy in a context of high ionizing doses. However, this question is also liable to arise for any other exposure to high ionizing doses, equivalent to those used in radiotherapy.

A number of clinical criteria are used by practitioners to quantify the tumor response following an anticancer treatment. Most of these criteria relate to the reduction in the tumor volume (or local control) after treatment or to the radiation dose to reduce this volume. However, none of these criteria are consensual.

The TCD50 and TCD95 (Tumor Control Dose) criteria represent the doses for which 50% and 95% tumor control are obtained, respectively. Frequently used in the 1980s when standard treatments allowed some standardization for a given tumor type, this is no longer the case for cancer diseases which are treated differently according to the centers with a wide variety in the dose spread.

The TCP (Tumor Control Probability) criterion makes it possible to predict theoretically the probability of tumor control. This criterion is based on radiobiological studies taking into account the clonogenic survival of tumor cells irradiated in vitro.

Mathematically, TCP is based on a Linear-Quadratic model:

TCP=exp^{-N0exp(γT-αD-βD2)}

where α and β are the linear quadratic model parameters, γ the cell proliferation constant and T the treatment time in days.

The RECIST (Response Evaluation Criteria In Solid Tumors) criterion is one of the most commonly used criteria at the present time to describe the progression of the solid tumor after radiotherapy treatment. It was published in February 2000 by an international collaborative project and revised in 2009 (Eisenhauer et al., “New response evaluation criteria in solid tumours: Revised RECIST guideline (version 1.1)”, European J Cancer 45 (1009), p. 228-247). This criterion is based on the reduction in the sum of the largest tumor diameters:
Complete Response (CR): complete tumor disappearance
Partial Response (PR): at least 30% reduction in the sum of the largest tumor diameters
Stable Disease (SD): no noteworthy change
Progressive Disease (PD): at least 20% increase in the sum of the largest tumor diameters.

Furthermore, two imaging criteria are used by clinicians, particularly criteria relating to the variation in signal intensity after radiotherapy treatment. This intensity is generally measured by a PetScan or infusion MRI. This intensity may be correlated with a number of surviving cells since only the part of the tumor containing living cells will emit a signal as this part will still be vascularized. Moreover, the Choi criterion makes it possible to correlate the tumor volume with the signal intensity (Choi et al 2007, “Correlation of Computed Tomography and Positron Emission Tomography in Patients With Metastatic Gastrointestinal Stromal Tumor Treated at a Single Institution With Imatinib Mesylate: Proposal of New Computed Tomography Response Criteria” J Clin Oncol 25.1753-1759.).

Those skilled in the art also know the cellular parameters for describing tumors.

One of these parameters is cell survival: in 1956, Puck and Marcus proposed using the in vitro colony test to quantify radiosensitivity: after inoculating a known number of irradiated cells, the measurement of the number of colonies (macroscopic cluster of proliferative surviving cells after 5-6 generations) formed by the cells that radiation had not sterilized becomes the reference number for evaluating the surviving fraction of cells irradiated in vitro. The survival curves are defined by representing in a semi-logarithmic graph the dose (x-axis)−survival (logarithmic y-axis) relationship by a series of tests at different doses, each delivered generally in a single fraction. These curves are described using the Linear-Quadratic model:

SF(D)=exp(−αD−βD²) where α and β are adjustment parameters.

This formula is valid for single-dose radiation. However, if fractionated radiation is taken into consideration, i.e. a total dose D divided into n fractions of a dose d, this gives:

D=n×d

Sd(d)=exp(−αd−βd2)

This dose will be repeated n times, to reach a total treatment dose D.

$$\begin{matrix}SF ( D , d ) = SF ( d ) n \\ = exp ( - α d - β d 2 ) n \\ = exp ( - n α d - n β d 2 ) \\ = exp ( - α D - β d D )\end{matrix}$$

In the case of dose fractionation, the survival will be described using the following formula:

SF(d,D)=exp(−αD−βdD)

where d is the dose in Gy per fraction

The survival curve for single-dose radiation, respectively for radiation with a total dose D divided into n fractions of a dose d is represented in FIG. 1A, respectively in FIG. 1B:

As such, as seen in FIG. 1, single-dose radiation induces greater cell damage than fractionated radiation for the same total treatment dose. This type of fractionation, though it lowers the efficacy of the treatment on the tumor, makes it possible to reduce radiotherapy side-effects.

More advanced treatment techniques, such as the cyberknife and tomotherapy, make it possible to deliver a greater dose per fraction to the tumor while sparing healthy tissue, rendering the treatment more effective.

The surviving fraction at 2 Gy (SF2) is frequently used to predict the radiosensitivity of a tumor. A linear relationship was found between TCD95 and SF2 by Fertil and Malaise in 1981 (see the publication cited above):

TCD95=142.8×SF2+8.57

Several research teams have attempted to correlate SF2 with the different clinical tumor response criteria cited above, but this has not led to a general, relatively simply model for developing predictive tests.

A further parameter is the number of surviving cells: the number of surviving cells after radiation is a quantifiable parameter used to define tumor control and volume reduction. It will be directly correlated with cell survival and the number of initial cells in the tumor N0:

N=N0SF(d,D)

In molecular terms, the prior art reports analyses using the H2AX or pH2AX marker for predicting radiotherapy efficacy. As such, Mahrhofer et al. (“Radiation induced DNA damage and damage repair in human tumour and fibroblast cell lines assessed by histone HAX phosphorylation”, Int J Oncol Biol Phys, 2006. 64(2): p. 573-80) demonstrated on five cancer lines that there was no correlation between the marker pH2AX and tumor radiosensitivity defined by the clonogenic survival at 2 Gy (SF2). However, their study does not account for the number of actual pH2AX foci (standardization of results) and it is based only on SF2. Similarly, Kock et al. (“Residual yH2AX foci predict local tumour control after radiotherapy”, Radiotherapy and Oncology, 2013, 108. p. 434-9) proposed a model based on correlation between the number of standardized pH2AX foci and TCD50. However, this study was conducted on xenografts, which does not reflect the physiological reality of the tumor.

The patent application EP 2 446 310 A1 (Helmholtz Zentrum Munchen) describes the repair of DNA double-strand breaks in the presence of the phosphorylated form of histone H2AX (known as gamma-H2AX or g-H2AX).

The patent applications US 2008/234946 and US 2012/041908 (University of South Florida et al.) describe a method for predicting the radiosensitivity of cancer cells, and not healthy cells; furthermore, it is based on genomic data and not on functional tests.

The patent application WO2014/154854 (Montpellier University Hospital Center) describes a method for predicting the radiosensitivity of a subject via the use of at least one radiosensitivity biomarker. This method does not detect markers directly linked with DNA damage and repair; furthermore, it is based on proteomics data. Furthermore, this patent application does not describe a quantitative relationship between radiobiological data and the severity of tissue reactions.

The patent application WO 2013/187973 (University of California) describes systems and methods for determining the radiosensitivity of cells and/or of a subject in relation to a control population. More specifically, this method includes the radiation of a biological specimen, the detection and quantification of radiation-induced foci in erythrocyte cells, lymphocytes or primary cells, resulting from a blood sample via the use of one or a plurality of detection markers from a set of markers including anti-pH2AX, anti-MRE11 and anti-ATM. The quantification of radiation-induced foci at different post-radiation observation times less than 2 hours makes it possible to determine the repair kinetics thereof which is correlated empirically with the subject's radiosensitivity. However, the analysis of foci in lymphocyte type cells is very difficult due to the small nucleus thereof. Furthermore, this method does not however allow the practitioner to make decisions in respect of the patient's treatment.

The patent application WO 2010/88650 (University of Texas) describes methods and compositions for identifying cancer cells which are either sensitive or resistant to a specific radiotherapy treatment; therefore, it is not applicable to any radiotherapeutic treatment.

The patent application WO 2010/109357 describes a method and an apparatus for adaptive radiotherapy protocol planning based on optimizing the probability of normal tissue complication and the probability of tumor control according to specific markers for each patient. The values of the markers associated with normal tissues comprise in vitro test results, protein mass spectrometry signatures, patient previous medical history and record data. The in vitro test values may be of cellular, proteomic and genetic origin such as, without being restricted thereto, various counts of cells, HB, CRP, PSA, TNF-alpha, ferritin, transferrin, LDH, IL-6, hepcidin, creatinine, glucose, HbA1c, and telomer length. The markers from the patient's previous medical history and records include previous abdominal surgery, hormonal medication or anticoagulants, diabetes, age, and tumor growth-related measurements, such as biomarkers associated with various forms of ablation. However, the individual radiosensitivity is not taken into account therein.

Despite this extensive prior art, the applicant has observed that the patents do not describe a method for quantifying tumor sensitivity to a DNA-breaking treatment suitable for evaluating quantitatively the efficacy of an anti-cancer treatment, which may be used for any patient and any type of treatment liable to induce DSBs, and particularly for any type of ionizing radiation. The problem of providing a predictive method of the tumor sensitivity and predicting tumor control regardless of the clinical parameters used therefore remains without an operational solution. The aim of the present invention is that of proposing a novel predictive method of tumor sensitivity to a DNA-breaking treatment.

### SUMMARY

The invention relates to the field of medical radiotherapy, and more particularly the field of radiotherapeutic laboratory methods. The invention relates to a novel predictive method of cellular, tissue and clinical radiosensitivity, which is based on the determination and cross-checking of a plurality of cellular and enzymatic parameters and criteria applied to the tumor response. More particularly, the invention relates to a predictive method for characterizing the sensitivity of a tumor in response to a DNA-breaking radiotherapy treatment.

The inventors found a correlation between cell survival at a dose D and a dose fractionation d and the number of foci of pH2AX, that is:

$$SF ( d , D ) = {e}^{- ( \frac{n \times {N}_{H 2 AX} ( 2 , 24 h )}{θ} + β d D )}$$

Where NH2AX represents the number of foci of pH2AX in surviving tumor cells 24 hours post-radiation, θ represents the cell tolerance (unit: number of double-strand breaks), D is the dose in Gy, d is the dose in Gy per fraction, n is the number of fractions, and β: parameter in Gy⁻² of the linear quadratic model relative to the tumor tissue type, in the knowledge that θ and β are decimal or integer adjustment parameters, preferably decimal adjustment parameters corresponding to the arithmetic rounding of the value obtained by calculation, preferably, with two significant digits after the decimal point, more preferentially with three significant digits after the decimal point, even more preferentially with four significant digits after the decimal point.

On the basis of this observation, the inventors found correlations between clinical parameters and some molecular and/or cellular parameters.

For the parameter TCD95:

1) According to the number of surviving cells:

$$TCD 95 = A \times \frac{N ( 2 Gy )}{{N}_{0}} + B$$

where A is an integer or decimal constant between 130 Gy and 160 Gy, and B is an integer or decimal constant between 5 Gy and 15 Gy.

Preferably, the parameter TCD95 is determined according to the following formula:

$$TCD 95 = 142.8 \times \frac{N ( 2 Gy )}{{N}_{0}} + 8.57$$

2) According to the number of pH2AX foci (this number being referred to as NH2AX)

$$TCD 95 = A \times {e}^{- ( \frac{{N}_{H 2 AX} ( 2 Gy , 24 h )}{θ} + 4 β )} + B$$

where A is an integer or decimal constant between 130 Gy and 160 Gy, and B is an integer or decimal constant between 5 Gy and 15 Gy.

Preferably, the parameter TCD95 is determined according to the following formula:

$$TCD 95 = 142.8 \times {e}^{- ( \frac{{N}_{H 2 AX} ( 2 Gy , 24 h )}{θ} + 4 β )} + 8.57$$

In the knowledge that θ and β are adjustment parameters as explained above.

For the parameter TCP:

1) According to the number of surviving cells after treatment with a dose D:

TCP(D)=e−N(D)

In the knowledge that this correlation is inferred from the following known correlation (Zainer and Minerbo, “Tumour control probability: a formulation applicable to any temporal protocol of dose delivery”, Phys Med Biol. 2000 February; 45(2):279-93):

TCP(D)=e−N0SF(D)

2) According to the number of pH2AX foci (this number being referred to as NH2AX)

$$TCP ( D ) = {e}^{- {N}_{0} \times {e}^{- ( \frac{n \times {N}_{H 2 AX} ( d , 24 h )}{θ} + β dD )}}$$

In the knowledge that θ and β are adjustment parameters as explained above.

The inventors observed that these parameters of TCD95 and TCP as determined according to the invention indeed give a more precise predictive description than the methods according to the prior art, but that the predictive description based on cellular and/or molecular parameters may be improved significantly if the sensitivity of the tumor in response to a DNA-breaking treatment is characterized by evaluating the surviving cellular fraction at a dose D and a dose fractionation d, or by the tumor volume. This embodiment also has the advantage that:
SF(d,D) may be easily determined on the basis of the number of foci of pH2AX
the volume being a geometric factor, it may be correlated with all the other clinical criteria used by practitioners according to the prior art to describe tumor status and progression, such as the RECIST criterion, mentioned above.

For the surviving cell fraction at a dose D

The inventors also found a correlation between the number of pH2AX foci at 24 hours and cell survival. For a dose of 2Gy, the cell survival is:

$$SF ( 2 Gy ) = {e}^{- ( \frac{{N}_{H 2 AX} ( 2 Gy , 24 h )}{θ} + 4 β )}$$

or for a dose D and a dose fractionation d:

$$SF ( d , D ) = {e}^{- ( \frac{n \times {N}_{H 2 AX} ( d , 24 h )}{θ} + β dD )}$$

where: NH2AX represents the number of foci of pH2AX in surviving tumor cells 24 hours post-radiation, θ represents the cell tolerance (unit: number of double-strand breaks), D is the dose in Gy, d is the dose in Gy per fraction, n is the number of fractions, and β represents the parameter in Gy-2 of the linear quadratic model relative to the tumor tissue type.

In the knowledge that θ and β are adjustment parameters as explained above.

For the tumor volume:

1) According to survival:

$$V ( D ) = \frac{{V}_{0}}{1 + {e}^{- a , {N}_{0} ( SF ( D ) - N ( {D}_{50} ) )}}$$

In the knowledge that V(D) expresses the tumor volume surviving a dose D.

2) According to the number of surviving cells:

$$V ( D ) = \frac{{V}_{0}}{1 + {e}^{- a ( N ( D ) - N ( {D}_{50} ) )}}$$

3) According to the number of pH2AX foci

$$V ( d , D ) = \frac{{V}_{0}}{1 + {e}^{- a , {N}_{0} ( {e}^{- ( \frac{n \times {N}_{H 2 AX} ( d , 24 h )}{θ} + β dD )} - {e}^{- ( \frac{n 50 \times {N}_{H 2 AX} ( d , 24 h )}{θ} + β dD 50 )} )}}$$

In these formulas linked with the tumor volume:

N0 represents the number of initial tumor cells,

θ represents the cell tolerance (unit: number of double-strand breaks),

D50 is the dose for which 50% tumor volume reduction is observed,

n50 is the number of fractions for which 50% tumor volume reduction is observed,

a is a volume variation constant per number of breaks,

β is a parameter (in Gy-2) of the linear quadratic model relative to the tumor tissue type.

In the knowledge that θ and β are adjustment parameters as explained above.

Furthermore, this surviving fraction at a dose D (fractionated into n doses d) is proportional to the intensity ratio of the signals collected by imaging before and after radiotherapy treatment according to the formula:

$$\frac{{I}_{f}}{{I}_{i}} = c \times SF ( d , D ) = c \times {e}^{- ( \frac{n \times {N}_{H 2 AX} ( d , 24 h )}{θ} + β dD )}$$

where:

If is the signal intensity in the treated volume at the end of treatment,

Ii is the signal intensity in the treated volume before treatment,

C is a proportionality constant.

All these correlations according to the invention may be used for an ionizing radiation dose. In one embodiment, the dose d is the dose per radiotherapeutic treatment fraction (typically 2 Gy) and D is the total dose of the radiotherapeutic treatment administered.

As such, the present invention relates to a process for evaluating the response of a tumor to a DNA-breaking treatment using a sample of cells from said tumor (preferably by biopsy), wherein:

(a) a cell sample is prepared from the cells taken from said tumor;

(b) said cell sample is subjected to a DNA-breaking treatment characterized by a dose D;

(c) the mean number of nuclear foci obtained with a marker pH2AX at the observation times t (these mean numbers being respectively called NpH2AX(t)) is determined on said cell sample, said observation times t being the time t=0 min (called t0, state before administration of said dose D) and at least one observation time selected from t=t1, t2, t3 and t4 after administration of said absorbed dose D;

(d) at least one parameter or score which makes it possible to characterize the response of the sample to said DNA-breaking treatment is determined, using at least the mean numbers NpH2AX(t),

and in which process:
t4 is a fixed value which represents the time for which the level of DNA breaks reaches its residual value, and which is advantageously chosen between 6 times t3 and 8 times t3, but must in this case be at least 12 hours, and preferably between 12 hours and 48 hours, and which is even more preferentially approximately 24 hours;
t3 is a fixed value which represents the time after which approximately 25% of the double-strand breaks (DSBs) are repaired in control cells from radioresistant patients, and which is advantageously chosen between 3 times t2 and 5 times t2, but must in this case be at least 2.5 hours and at most 6 hours, and is preferably between 3 hours and 5 hours, and is even more preferentially approximately 4 hours;
t2 is a fixed value which represents the time after which approximately 50% of the DSBs are repaired in control cells from radioresistant patients, and which is advantageously chosen between 5 times t1 and 7 times t1, but which must in this case be at least 35 minutes and at most 90 minutes, and is preferably between 45 minutes and 75 minutes, and is even more preferentially approximately 60 minutes;
t1 is a fixed value which represents the time after which the number of recognized DSBs reaches its maximum in control cells from radioresistant patients, and which is advantageously chosen between 5 minutes and 15 minutes after discontinuing radiation, preferably between 7.5 minutes and 12.5 minutes, and even more preferentially at approximately 10 minutes.

Said DNA-breaking treatment is radiation with ionizing radiation.

Said tumor may be a solid or liquid tumor.

In one embodiment of the process according to the invention, the mean numbers N_{pH2AX}(t) are determined at t=t0, t1, t2, t3 and t4.

In a further embodiment, at least one parameter selected in the group formed by: the surviving cell fraction after a dose D fractionated into n doses d (SF(d,D)), the parameter TCD50, the parameter TCD95, the parameter TCP, the tumor volume, is determined as a parameter or score of the response of the sample to said DNA-breaking treatment.

In the case where said DNA-breaking treatment is ionizing radiation, the parameter TCD95 is advantageously defined using the number of cells surviving the DNA-breaking treatment with a dose D, preferably by means of the relation

$$TCD 95 = A \times \frac{N ( 2 Gy )}{{N}_{0}} + B$$

where N0 represents the number of initial tumor cells, N(2 Gy) represents the number of surviving cells at t4 after DNA-breaking treatment radiation with ionizing radiation at a dose of 2 Gy, said dose being preferably an ionizing radiation dose between 0.5 Gy and 5 Gy, preferably between 1 Gy and 3 Gy, and even more preferentially 2 Gy, A is an integer or decimal constant between 13 Gy and 160 Gy, and

B is an integer or decimal constant between 5 Gy and 15 Gy.

In the case where said DNA-breaking treatment is ionizing radiation, the parameter TCD95 is advantageously defined using the number of cells surviving the DNA-breaking treatment with a dose D, preferably by means of the relation

$$TCD 95 = 142.8 \times \frac{N ( 2 Gy )}{{N}_{0}} + 8.57$$

where N0 represents the number of initial tumor cells, and N(2 Gy) represents the number of surviving cells at t4 after DNA-breaking treatment radiation with ionizing radiation at a dose of 2 Gy, said dose being preferably an ionizing radiation dose between 0.5 Gy and 5 Gy, preferably between 1 Gy and 3 Gy, and even more preferentially 2 Gy.

In the case where said DNA-breaking treatment is ionizing radiation, the parameter TCD95 is defined using the number of pH2AX foci of cells surviving the DNA-breaking treatment with a dose D, preferably by means of the relation:

$$TCD 95 = A \times {e}^{- ( \frac{{N}_{H 2 AX} ( 2 Gy , 24 h )}{θ} + 4 β )} + B$$

where NH2AX(2Gy, 24 h) represents the number of pH2AX foci at the time t4 after a DNA-breaking treatment dose D, said dose being preferably an ionizing radiation dose of 2 Gy, θ represents the cell tolerance (unit: number of double-strand breaks), β represents the parameter in Gy-2 of the linear quadratic model relative to the tumor tissue type, A is an integer or decimal constant in Gy between 130 Gy and 160 Gy, and B is an integer or decimal constant in Gy between 5 Gy and 15 Gy.

In the same case, the parameter TCD95 may be defined using the number of pH2AX foci of cells surviving the DNA-breaking treatment with a dose D, preferably by means of the relation:

$$TCD 95 = 142.8 \times {e}^{- ( \frac{{N}_{H 2 AX} ( 2 Gy , 24 h )}{θ} + 4 β )} + 8.57$$

where NH2AX(2Gy, 24 h) represents the number of pH2AX foci at the time t4 after a DNA-breaking treatment dose D, said dose being preferably an ionizing radiation dose of 2 Gy, θ represents the cell tolerance (unit: number of double-strand breaks) and β the parameter in Gy-2 of the linear quadratic model relative to the tumor tissue type.

In the knowledge that θ and β are adjustment parameters as explained above.

In the same case, the parameter TCP may be defined using the number of surviving cells after DNA-breaking treatment with a dose D, preferably by means of the relation

TCP(D)=e−N(D)

where N represents the number of surviving cells after a DNA-breaking treatment dose D, said dose being preferably an ionizing radiation dose of 2 Gy.

In the same case, the parameter TCP may be defined using the number of pH2AX foci of cells surviving the DNA-breaking treatment with a DNA-breaking treatment dose D, preferably by means of the relation:

$$TCP ( D ) = {e}^{- {N}_{0} \times {e}^{- ( \frac{n \times {N}_{H 2 AX} ( d , 24 h )}{θ} + β dD )}}$$

where NH2AX(d, 24 h) represents the number of pH2AX foci in surviving cells at the time t4 after a DNA-breaking treatment dose d, said dose being preferably an ionizing radiation dose of 2 Gy,

In the knowledge that θ and β are adjustment parameters as explained above.

In one embodiment, the tumor cell survival after DNA-breaking treatment with a dose D fractionated into n doses d is defined preferably by means of the relation:

$$SF ( d , D ) = {e}^{- ( \frac{n \times {N}_{H 2 AX} ( d , 24 h )}{θ} + β dD )}$$

where: NH2AX represents the number of pH2AX foci in surviving tumor cells 24 hours post-radiation, θ represents the cell tolerance (unit: number of double-strand breaks), D is the dose in Gy, d is the dose in Gy per fraction, n is the number of fractions, and β is the parameter in Gy-2 of the linear quadratic model relative to the tumor tissue type.

The proportionality of said surviving cell fraction after a dose D fractionated into n doses d is defined with the intensity ratio of the signals collected by imaging before and after radiotherapy treatment according to the formula:

$$\frac{{I}_{f}}{{I}_{i}} = c \times SF ( d , D ) = c \times {e}^{- ( \frac{n \times {N}_{H 2 AX} ( d , 24 h )}{θ} + β dD )}$$

where:

If is the signal intensity in the treated volume at the end of treatment,

Ii is the signal intensity in the treated volume before treatment,

C is a proportionality constant.

In one advantageous embodiment, the tumor volume after DNA-breaking treatment with a dose D (this volume being expressed by the parameter V(D)) is defined using the tumor cell survival, preferably by means of the relation

$$V ( D ) = \frac{{V}_{0}}{1 + {e}^{- a , {N}_{0} ( SF ( D ) - SF ( {D}_{50} ) )}}$$

where:
N is the number of surviving cells after radiation with a dose D,
V0 is the initial tumor volume,
SF(D) is the surviving cell fraction at a dose D,
a is a volume variation constant per number of DNA breaks,
D50 is the dose in Gy for which 50% tumor volume reduction is observed.

In a further advantageous embodiment, the tumor volume after DNA-breaking treatment with a dose D (this volume being expressed by the parameter V(D)) is defined using the tumor cell survival, preferably by means of the relation

$$V ( D ) = \frac{{V}_{0}}{1 + {e}^{- a ( N ( D ) - N ( {D}_{50} ) )}}$$

where:
N is the number of surviving cells after radiation with a dose D,
V0 is the initial tumor volume,
N(D) is the number of surviving cells at a dose D,
a is a volume variation constant per number of DNA breaks,
D50 is the dose in Gy for which 50% tumor volume reduction is observed.

In a further embodiment, the tumor volume after DNA-breaking treatment at a dose D fractionated into n doses d is defined, using the number of pH2AX foci of cells surviving the DNA-breaking treatment, preferably by means of the relation

$$V ( d , D ) = \frac{{V}_{0}}{1 + {e}^{- a , {N}_{0} ( {e}^{- ( \frac{n \times {N}_{H 2 AX} ( d , 24 h )}{θ} + β dD )} - {e}^{- ( \frac{n 50 \times {N}_{H 2 AX} ( d , 24 h )}{θ} + β dD 50 )} )}}$$

where:

N0 represents the number of initial tumor cells,

θ represents the cell tolerance (unit: number of double-strand breaks),

D50 is the dose for which 50% tumor volume reduction is observed,

n50 is the number of fractions for which 50% tumor volume reduction is observed,

a is a volume variation constant per number of breaks,

β represents the parameter in Gy-2 of the linear quadratic model relative to the tumor tissue type,

In the knowledge that θ and β are adjustment parameters as explained above.

### DRAWINGS

FIG. 1 represents the variation of cell survival after single-dose radiation (see FIG. 1A) and the variation of cell survival for radiation with a total dose D divided into n fractions of a dose d (see FIG. 1B): FIG. 1 shows that single-dose radiation is much more lethal on a cellular level than fractionated radiation for the same total treatment dose. This type of fractionation, though it lowers the efficacy of the treatment at tumor level, makes it possible to reduce radiotherapy-related side-effects.

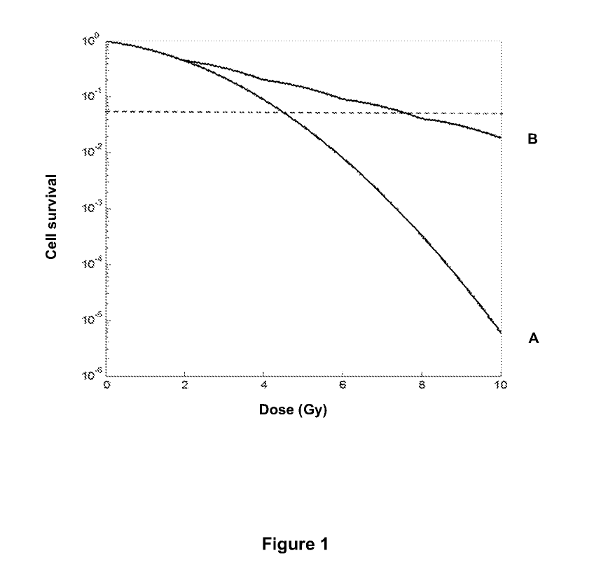

FIG. 2 represents the variation of the cell survival fraction at a single dose of 2 Gy (SF2(%)) according to the number of pH2AX foci acquired per cell, after 24 hours of post-radiation repair time with an absorbed dose of 2 Gy. Each point corresponds to the variation of cell survival at 2 Gy according to the number of pH2AX foci acquired per cell, after 24 hours of post-radiation repair time with an absorbed dose of 2 Gy for a cell line. A simulation according to the invention correlating SF2(%) with the number of pH2AX foci acquired per cell, after 24 hours of post-radiation repair time with an absorbed dose of 2 Gy is represented as a dotted line in FIG. 2.

### DESCRIPTION

Within the scope of the present invention, the DNA-breaking treatment is quantified by the dose D thereof. In the case where said DNA-breaking treatment is ionizing radiation (radiotherapy), said dose D corresponds to the absorbed dose of said ionizing radiation (commonly expressed in Gy).

An embodiment with a plurality of alternative embodiments suitable for a human patient is described herein.

Test Preparation

Before sampling any cells and before handling any sampled cells, the respective operators (belonging for example to a cytological analysis laboratory) are informed (typically by a physician) of the patient's potential HIV or hepatitis C infection status so that the operators can take suitable increased biological safety measures when sampling, handling and managing the cell culture.

Then, the operator takes a tumor sample from the patient. The cell sample is placed in sterile DMEM medium+20% fetal calf serum. The sample is transferred without delay to a specialized laboratory, in the knowledge that the sample must not remain more than 38 hours at ambient temperature.

The following step represents the isolation and/or amplification of the sampled tissue.

In one embodiment, on receipt, the cell sample (typically the biopsy) is established in the form of an amplifiable cell line, very preferably via a selective culture by means of flow cytometry or via the use of selective culture media, without a viral or chemical transformation agent according to a well-known modulable ancillary procedure according to the tumor type (Krönig et al, “Cell type specific gene expression analysis of prostate needle biopsies resolves tumor tissue heterogeneity” Oncotarget. 2015 Jan. 20; 6(2):1302-14; Hristozova et al, “A simple multicolor flow cytometry protocol for detection and molecular characterization of circulating tumor cells in epithelial cancers” Cytometry A. 2012 June; 81(6):489-95. doi: 10.1002/cyto.a.22041. Epub 2012 Mar. 21; Wang et al, “Identification and characterization of cells with cancer stem cell properties in human primary lung cancer cell lines” PLoS One. 2013; 8(3):2013 Mar. 4.). Once the number of cells is sufficient (1-3 weeks), the cells are inoculated on glass coverslips in Petri dishes. A portion of these slides is irradiated on a medical radiation apparatus according to a certified dosimetry with an absorbed dose D (for example 2 Gy). A further portion is not irradiated; it represents the spontaneous state (absorbed dose 0 Gy).

The radiation may be performed for example with a medical accelerator which delivers 6 MV photons with an absorbed dose rate of 3 Gy min-1. After radiation and to undergo the repair times mentioned hereinafter, the cells remain in the culture incubator at 37° C.

For the irradiated cells, characteristics corresponding to the radiation-induced state after a plurality of repair times (post-radiation repair times) are acquired. At least two and even more preferentially at least three points are acquired, namely: t1, t2, t3 and t4. Said characteristics are represented by the foci corresponding to the marker pH2AX.

The cells on glass coverslips are then fixed, lysed and hybridized. The following procedure, known per se (see the cited publication by Bodgi et al.), may be used: the cells were fixed in 3% paraformaldehyde and 2% sucrose for 15 minutes at ambient temperature and permeabilized in 20 mM HEPES buffer solution (4-(2-hydroxyethyl)-1-piperazine ethane sulfonic acid) at pH 7.4, 50 mM NaCl, 3 mM MgCl2, 300 mM sucrose, 0.5% Triton X-100 (a non-ionic surfactant having the formula t-Oct-C6H4-(OCH2CH2)xOH where x=9-10, CAS No. 9002-93-1, supplied by Sigma Aldrich) for 3 minutes. The glass coverslips were then washed in phosphate buffer saline (known as the acronym PBS) before immunological staining. The incubation took place for 40 min at 37° C. in PBS supplemented with 2% bovine serum albumin (known as the acronym BSA or fraction V, supplied by Sigma Aldrich) and was followed by a wash with PBS. Anti-pH2AX primary antibodies were used at a concentration of 1:800. The incubations with anti-mouse FITC or anti-rabbit TRITC secondary antibodies (1:100, supplied by Sigma Aldrich) were performed at 37° C. in 2% BSA for 20 minutes. Glass coverslips were treated with Vectashield™ containing DAPI (4,6-Diamidino-2-phenylindole) to label the nucleus. Staining with DAPI also makes it possible, indirectly, to determine the number of cells in phase G1 (nuclei with homogenous DAPI staining), in phase S (nuclei with numerous pH2AX foci), in phase G2 (nuclei with heterogeneous DAPI staining) and metaphases (visible chromosomes).

The results are acquired using these coverslips on an immunofluorescence microscope (Olympus model for example). The reading may be direct (typically by counting the foci on at least 50 cells in G0/G1 for each point) or using dedicated image analysis software, or on an automated microscope; preferably the automated software or microscope methods are calibrated with manual determinations.

In order to obtain results of sufficient statistical reliability to serve as a basis for diagnosis, not more than 3 independent series of experiments (radiation) are performed and the mean of each of the numbers of foci for the times defined is calculated.

Determination of Biological and Clinical Parameters

General and Markers Used

The invention is based, inter alia, on the use of data acquired for the marker pH2AX on non-irradiated (spontaneous state) and irradiated (radiation-induced state) cells. The method is based on the kinetic study of labelling using this marker according to the repair time: the samples are labeled after a predetermined time interval from discontinuing radiation, and the immunofluorescence thereof is studied. The complete kinetic curves, for example represented by 5 points situated advantageously at t0, t1 (preferably 10 minutes), t2 (preferably 1 hr), t3 (preferably 4 hrs) and t4 (preferably 24 hrs) may be measured, in the knowledge that t0 corresponds to the state before radiation (spontaneous state).

However, the applicant realized that certain points (corresponding to certain repair times) are more important than others, and that some points are not predictive. Through suitable selection of the predetermined parameters at given times, it is thereby possible to reduce the number of measurements and therefore reduce the overall cost of diagnosis, without decreasing the predictive power of the method.

The means of each point and each dose with each marker are calculated with the standard errors of the mean (referred to as “SEM”) given that the sampling is n=3 (no Gaussian type “standard error SE”).

(i) pH2AX denotes the phosphorylated forms in serine 439 of variant X of histone H2AX marking, according to the applicant's observations, the number of DNA double-strand breaks (DSBs) that are recognized by the main and faithful repair mode, suture. The marker pH2AX is essentially nuclear in the form of nuclear foci only and only the number and size of the foci shall be analyzed.

Counterstaining with DAPI (a DNA marker known to those skilled in the art) makes it possible to locate the nucleus to situate the nuclear location.

Biological Parameters

The definition and determination are performed as indicated of:
NpH2AX(t), the mean number of nuclear foci obtained with the marker pH2AX, at the observation times t0 (non-irradiated) or t1, t2, t3, t4 after radiation (absorbed dose: 2 Gy), in the knowledge that the determination of the parameter NpH2AX(t) is mandatory within the scope of the method according to the invention.

Predictive Evaluation

This targets the prediction of clinical and radiotherapeutic parameters using the biological data measured. Several diagnostic levels are proposed:

A) A quantitative diagnosis directly derived from the mathematical value of the scores or mathematical formulas

This targets the prediction of clinical and radiotherapeutic parameters using the biological data measured. Several diagnostic levels are proposed:
The parameter TCD95:

$$TCD 95 = 142.8 \times {e}^{- ( \frac{{N}_{H 2 AX} ( 2 Gy , 24 h )}{θ} + 4 β )} + 8.57$$

For the parameter TCP:

$$TCP ( D ) = {e}^{- {N}_{0} \times {e}^{- ( \frac{n \times {N}_{H 2 AX} ( d , 24 \cdot h )}{θ} + β dD )}}$$

For the surviving cell fraction at a dose D and at a dose fractionation d:

$$SF ( d , D ) = {e}^{- ( \frac{n \times {N}_{H 2 AX} ( d , 24 \cdot h )}{θ} + β dD )}$$

where: NH2AX represents the number of pH2AX foci in surviving tumor cells 24 hours post-radiation, θ represents the cell tolerance (unit: number of double-strand breaks), D is the dose in Gy, d is the dose in Gy per fraction, n is the number of fractions, and β represents the parameter in Gy-2 of the linear quadratic model relative to the tumor tissue type.

In the knowledge that θ and β are adjustment parameters as explained above.

Furthermore, this surviving fraction at a dose D (fractionated into n doses d) is proportional to the intensity ratio of the signals collected by imaging before and after radiotherapy treatment according to the formula:

$$\frac{{I}_{f}}{{I}_{i}} = c \times SF ( d , D ) = c \times {e}^{- ( \frac{n \times {N}_{H 2 AX} ( d , 24 \cdot h )}{θ} + β dD )}$$

where:

If is the signal intensity in the treated volume at the end of treatment,

Ii is the signal intensity in the treated volume before treatment,

C is a proportionality constant.

In the knowledge that θ and β are adjustment parameters as explained above.
For the tumor volume:

$$V ( d , D ) = \frac{{V}_{0}}{1 + {e}^{- a . {N}_{0} ( \begin{matrix}{e}^{- ( \frac{n \times {N}_{H 2 AX} ( d , 24 \cdot h )}{θ} + β dD ) -} \\ {e}^{- ( \frac{n 50 \times {N}_{H 2 AX} ( d , 24 \cdot h )}{θ} + β dD 5 \; 0 )}\end{matrix} )}}$$

In these formulas linked with the tumor volume:

N0 represents the number of initial tumor cells,

θ represents the cell tolerance (unit: number of double-strand breaks),

D50 is the dose for which 50% tumor volume reduction is observed,

n50 is the number of fractions for which 50% tumor volume reduction is observed,

a is a volume variation constant per number of breaks,

β represents the parameter in Gy-2 of the linear quadratic model relative to the tumor tissue type,

In the knowledge that θ and β are adjustment parameters as explained above.

B) a more qualitative diagnosis, influenced by the quantitative diagnosis but accounting for any clinical data brought to the practitioner's knowledge.

Analysis Levels and Methods

In a first embodiment, all or part of the values NpH2AX(t) determined in an algorithm are used, resulting in a parameter or score suitable for characterizing the response of the sample to said dose D of said DNA-breaking treatment.

In a second embodiment, the observation that some values are not predictive for any score is taken into account: this is the case for example of points NpH2AX(t3). For this reason, it is possible to envisage a restricted analysis where only the points t0, t1, t2 and t4 are used among the values NpH2AX(t) determined.

### Example: Validation of the Equation According to the Invention Suitable for Determining the Survival Fraction at 2 Gy According to the Number of pH2AX Foci

The cell lines presented in table 1 were amplified according to the recommendations of the supplier (SIGMA-ALDRICH) until the number of cells sought was obtained. After obtaining a sufficient number of cells (generally after one to 3 weeks), the first experiments were conducted using the process according to the invention. The cells were inoculated on glass coverslips in Petri dishes. These coverslips were irradiated on a medical radiation apparatus according to a certified dosimetry with an absorbed dose D of 2 Gy.

The radiation was performed with a medical accelerator which delivers 6 MV photons with an absorbed dose rate of 3 Gy min-1. After radiation with an absorbed dose of 2 Gy, the cells were stored in the culture incubator at 37° C. The samples were then labeled after 24 hours of post-radiation repair, namely: 24 hours (t4) from discontinuing radiation, and the mean number of nuclear foci obtained with the marker pH2AX after 24 hours of post-radiation repair was acquired (see table 1).

The cells having undergone radiation were then fixed, lysed and hybridized on glass coverslips. The cells were fixed in 3% paraformaldehyde and 2% sucrose for 15 minutes at ambient temperature and permeabilized in 20 mM HEPES buffer solution (4-(2-hydroxyethyl)-1-piperazine ethane sulfonic acid) at pH 7.4, 50 mM NaCl, 3 mM MgCl2, 300 mM sucrose, 0.5% Triton X-100 (a non-ionic surfactant having the formula t-Oct-C6H4-(OCH2CH2)xOH where x=9-10, CAS No. 9002-93-1, supplied by Sigma Aldrich) for 3 minutes. The glass coverslips were then washed in phosphate buffer saline (known as the acronym PBS) before immunological staining. The incubation took place for 40 min at 37° C. in PBS supplemented with 2% bovine serum albumin (known as the acronym BSA or fraction V, supplied by Sigma Aldrich) and was followed by a wash with PBS. Anti-pH2AX primary antibodies were used at a concentration of 1:800. The incubations with anti-mouse FITC or anti-rabbit TRITC secondary antibodies (1:100, supplied by Sigma Aldrich) were performed at 37° C. in 2% BSA for 20 minutes.

The results (mean number of nuclear foci obtained with the marker pH2AX) were acquired using these coverslips on an immunofluorescence microscope (Olympus model for example). The reading was performed directly by counting the foci on at least 50 cells in G0/G1 for each point) or using dedicated image analysis software (imageJ).

In order to obtain results of sufficient statistical reliability to serve as a basis for diagnosis, 3 independent series of experiments were performed. The mean and standard errors of the mean (“SEM” or a) of each of the numbers of foci acquired after 24 hours of repair time after radiation with an absorbed dose of 2 Gy was calculated and presented in table 1.

SF2(%) was determined experimentally by conducting a clonogenic survival experiment at a dose of 2 Gy using adherent cell lines. These experiments were conducted according to a four-phase procedure well-known to those skilled in the art:

1) treatment of the cell monolayer in tissue culture flasks,

2) preparation of the simple cell suspensions and inoculation of a suitable number of cells in Petri dishes,

3) radiation at a dose D=2 Gy of these cells, and

4) fixing and staining of the colonies after a suitable incubation period, which may vary from 1-3 weeks, according to the cell line. The number of surviving cells was expressed as a % of survival.

The number of pH2AX foci after 24 hours of post-radiation repair time with an absorbed dose of 2 Gy and of the survival fraction at 2 Gy obtained are presented in table 1 hereinafter, for a plurality of tumor cell lines.

TABLE 1
Detection of the number of pH2AX foci (N_{H2AX}) after 24 hours of post-
radiation repair time with an absorbed dose of 2 Gy and of the survival
fraction at 2 Gy for a plurality of tumor cell lines.

| Cell line | Tumor type | SF2(%) | N_{H2AX}(24 h) |
|---|---|---|---|
| HT29 | colorectal carcinoma | 74 ± 2 | 7 ± 2 |
| Be11 | melanoma | 73 ± 1 | 3 ± 1 |
| SaOS2 | osteosarcoma | 73 ± 2 | 3 ± 1 |
| MCF7 | breast carcinoma | 68 ± 2 | 6 ± 2 |
| MO59K | glioblastoma | 65 ± 2 | 10 ± 2 |
| U2OS | osteosarcoma | 63 ± 3 | 9 ± 3 |
| Hela | cervix carcinoma | 60 ± 3 | 12 ± 2 |
| RT112 | bladder carcinoma | 60 ± 4 | 12 ± 2 |
| HRT18 | colorectal carcinoma | 54 ± 5 | 10 ± 2 |
| Ma11 | melanoma | 52 ± 2 | 6 ± 2 |
| IGROV | ovarian carcinoma | 52 ± 4 | 11 ± 3 |
| Na11 | melanoma | 51 ± 4 | 5 ± 4 |
| 2180 | nephroblastoma | 33 ± 4 | 15 ± 4 |
| SW48 | colorectal carcinoma | 22 ± 6 | 27 ± 3 |
| HCC1937 | ductal carcinoma | 22 ± 3 | 11 ± 2 |
| Hx142 | bladder carcinoma | 18 ± 4 | 14 ± 3 |
| CAPAN-1 | ovarian carcinoma | 10 ± 3 | 22 ± 5 |
| M059J | glioblastoma | 6 ± 5 | 29 ± 5 |

The data set presented in table 1 made it possible to determine the parameters β and 1/θ of the equation:

$$SF ( 2 Gy ) = {e}^{- ( \frac{{N}_{H 2 AX} ( 2 Gy , 24 h )}{θ} + 4 β )}$$

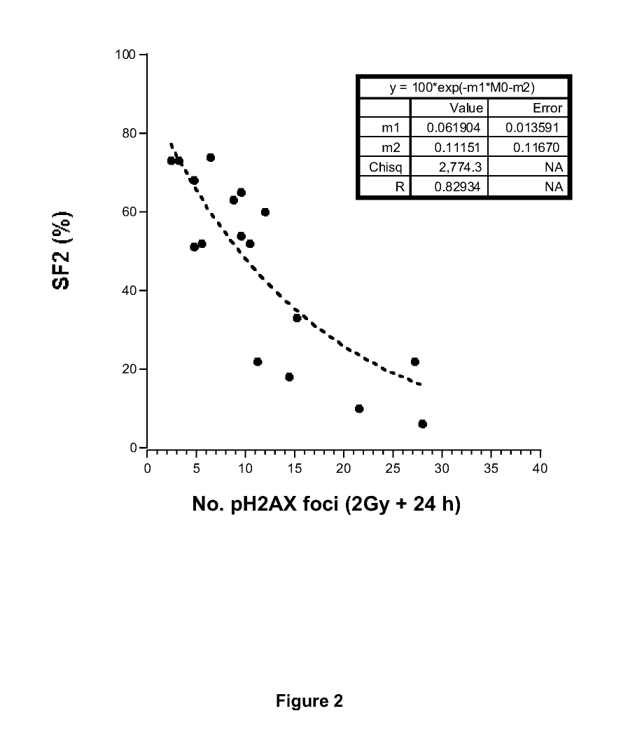

The variation of the cell survival fraction at 2 Gy (SF2(%)) according to the number of pH2AX foci acquired per cell (data from table 1), after 24 hours of post-radiation repair time with an absorbed dose of 2 Gy has been represented in FIG. 2. Each point corresponds to the variation of cell survival at 2 Gy according to the number of pH2AX foci acquired per cell, after 24 hours of post-radiation repair time with an absorbed dose of 2 Gy for a cell line. A simulation correlating SF2(%) with the number of pH2AX foci acquired per cell, after 24 hours of post-radiation repair time with an absorbed dose of 2 Gy is represented as a dotted line in FIG. 2.

The survival fraction for these tumor cell lines was simulated and expressed as a function of β and 1/θ.

SF2=exp(−0.0619*NH2AX(24h)−0.111) where R=0.82

where β=0.111/4=0.0277 and 1/θ=0.0619

The calculation of the values of β and 1/θ via the formula of SF2 makes it possible to obtain consistent results (these two numerical values indeed correspond to the mean values of β and 1/θ expected for tumors which are mostly group II) illustrating the different tumor radiosensitivities.

1-16. (canceled)

17. A method for evaluating a response of a tumor to a DNA-breaking treatment, the method comprising:
preparing a cell sample from cells taken from said tumor;
subjecting said cell sample to the DNA-breaking treatment at a dose D;
determining a mean number N_{pH2AX}(t) of nuclear foci obtained with a marker pH2AX at observation times on said cell sample, said observation times being time t=0 min and at least one observation time selected from t=t1, t2, t3 and t4 after administration of said absorbed dose D; and
determining, using at least the mean numbers N_{pH2AX}(t), at least one parameter or score which makes it possible to characterize a response of the sample to said DNA-breaking treatment,
wherein:
t4 is a fixed value which represents a time for which a level of DNA breaks reaches its residual value;
t3 is a fixed value which represents a time after which approximately 25% of double-strand breaks (DSBs) are repaired in control cells from radioresistant patients;
t2 is a fixed value which represents a time after which approximately 50% of the DSBs are repaired in control cells from radioresistant patients;
t1 is a fixed value which represents a time after which a number of recognized DSBs reaches a maximum in control cells from radioresistant patients,
t4 is between 6×t3 and 8×t3, but is approximately 24 hours;
t3 is between 3×t2 and 5×t2, but is approximately 4 hours;
t2 is between 5×t1 and 7×t1, but is approximately 60 minutes; and
t1 is 10 minutes after discontinuing radiation.

18. The method of claim 17, wherein said DNA-breaking treatment comprises radiation with ionizing radiation.

19. The method of claim 17, wherein said tumor comprises a solid tumor or liquid tumor.

20. The method of claim 17, wherein the mean numbers N_{pH2AX}(t) are determined at t=t0, t1, t2, t3 and t4.

21. The method of claim 17, further comprising determining at least one parameter selected in the group consisting of: a surviving cell fraction after the dose D is fractionated into n doses d (SF(d,D)), the parameter TCD50, the parameter TCD95, the tumor volume, as a parameter or score of the response of the sample to said DNA-breaking treatment.

22. The method of claim 21, wherein the parameter TCD95 is determined using a number of cells surviving the DNA-breaking treatment with the dose D, via the relation:
$$TCD 95 = A \times \frac{N ( 2 Gy )}{{N}_{0}} + B$$
where N₀ is a number of initial tumor cells, N(2 Gy) is a number of surviving cells at t4 after DNA-breaking treatment radiation with ionizing radiation at a dose of 2 Gy, A is an integer or decimal constant between 13 Gy and 160 Gy, and B is an integer or decimal constant between 5 Gy and 15 Gy.

23. The method of claim 21, wherein the parameter TCD95 is determined using a number of cells surviving the DNA-breaking treatment with the dose D, via the relation:
$$TCD 95 = 142.8 \times \frac{N ( 2 Gy )}{{N}_{0}} + 8.57$$
where N₀ is a number of initial tumor cells, and N(2 Gy) is a number of surviving cells at t4 after DNA-breaking treatment radiation with ionizing radiation at a dose of 2 Gy.

24. The method of claim 21, wherein the parameter TCD95 is determined using a number of pH2AX foci of cells surviving the DNA-breaking treatment with a dose D, via the relation:
$$TCD 95 = A \times {e}^{- ( \frac{{N}_{H 2 AX} ( 2 Gy , 24 h )}{θ} + 4 β )} + B$$
where N_{H2AX}(2Gy, 24 h) is a number of pH2AX foci at the time t4 after a DNA-breaking treatment dose D, said dose being an ionizing radiation dose of 2 Gy, θ is a cell tolerance, β is a parameter in Gy⁻² of a linear quadratic model relative to the tumor tissue type, A is an integer or decimal constant in Gy between 130 Gy and 160 Gy, and B is an integer or decimal constant in Gy between 5 Gy and 15 Gy.

25. The method of claim 21, wherein the parameter TCD95 is determined using a number of pH2AX foci of cells surviving the DNA-breaking treatment with a dose D, via the relation:
$$TCD 95 = 142.8 \times {e}^{- ( \frac{{N}_{H 2 AX} ( 2 Gy , 24 h )}{θ} + 4 β )} + 8.57$$
where N_{H2AX}(2Gy, 24 h) is a number of pH2AX foci at the time t4 after a DNA-breaking treatment dose D, said dose being an ionizing radiation dose of 2 Gy, θ is a cell tolerance, and β is a parameter in Gy⁻² of a linear quadratic model relative to the tumor tissue type.

26. The method of claim 17, further comprising determining a parameter TCP using a number of surviving cells after the DNA-breaking treatment with the dose D, via the relation:
TCP(D)=e^{-N(D)}
where N is the number of surviving cells after the DNA-breaking treatment dose D, and the dose D being an ionizing radiation dose of 2 Gy.

27. The method of claim 17, further comprising determining a parameter TCP using a number of pH2AX foci of cells surviving the DNA-breaking treatment with the dose D, via the relation:
$$TCP ( D ) = A \times {e}^{- {N}_{0} \times {e}^{- ( \frac{n \times {N}_{H 2 AX} ( d , 24 \cdot h )}{θ} + β dD )}}$$
where N_{H2AX}(d, 24 h) is the number of pH2AX foci in surviving cells at the time t4 after the DNA-breaking treatment dose d, said dose D being an ionizing radiation dose of 2 Gy.

28. The method of claim 17, further comprising determining the tumor cell survival after DNA-breaking treatment with the dose D fractionated into n doses d via the relation:
$$SF ( d , D ) = {e}^{- ( \frac{n \times {N}_{H 2 AX} ( d , 24 \cdot h )}{θ} + β dD )}$$
where: N_{H2AX} is the number of foci of pH2AX in surviving tumor cells 24 hours post-radiation, θ is a cell tolerance, D is the dose in Gy, d is a dose in Gy per fraction, n is a number of fractions, and β is the parameter in Gy⁻² of the linear quadratic model relative to the tumor tissue type.

29. The method of claim 17, further comprising determining a proportionality of said surviving cell fraction after the dose D fractionated into n doses d with an intensity ratio of signals collected by imaging before radiotherapy treatment and after radiotherapy treatment, via the relation:
$$\frac{{I}_{f}}{{I}_{i}} = c \times SF ( d , D ) = c \times {e}^{- ( \frac{n \times {N}_{H 2 AX} ( d , 24 \cdot h )}{θ} + β dD )}$$
where I_{f} is a signal intensity in the treated volume at the end of treatment, Iᵢ is a signal intensity in the treated volume before treatment, and C is a proportionality constant.

30. The method of claim 17, further comprising determining a tumor volume V(D) after DNA-breaking treatment with the dose D using a tumor cell survival, via the relation:
$$V ( D ) = \frac{{V}_{0}}{1 + {e}^{- a . {N}_{0} ( SF ( D ) - SF ( {D}_{50} ) )}}$$
where N is the number of surviving cells after radiation with a dose D, V0 is the initial tumor volume, SF(D) is the surviving cell fraction at a dose D, a is a volume variation constant per number of DNA breaks, and D50 is a dose in Gy for which 50% tumor volume reduction is observed.

31. The method of claim 17, further comprising determining a tumor volume V(D) after DNA-breaking treatment with the dose D using a tumor cell survival, via the relation:
$$V ( D ) = \frac{{V}_{0}}{1 + {e}^{- a ( N ( D ) - N ( {D}_{50} ) )}}$$
where N is the number of surviving cells after radiation with a dose D, V0 is the initial tumor volume, N(D) is the number of surviving cells at a dose D, a is a volume variation constant per number of DNA breaks, and D50 is the dose in Gy for which 50% tumor volume reduction is observed.

32. The method of claim 17, further comprising determining a tumor volume V(d,D) after DNA-breaking treatment at the dose D fractionated into n doses d, using the number of pH2AX foci of cells surviving the DNA-breaking treatment, via the relation:
$$V ( d , D ) = \frac{{V}_{0}}{1 + {e}^{- a . {N}_{0} ( \begin{matrix}{e}^{- ( \frac{n \times {N}_{H 2 AX} ( d , 24 \cdot h )}{θ} + β dD ) -} \\ {e}^{- ( \frac{n 50 \times {N}_{H 2 AX} ( d , 24 \cdot h )}{θ} + β dD 5 \; 0 )}\end{matrix} )}}$$
where N₀ is a number of initial tumor cells, θ is a cell tolerance, n₅₀ is a number of fractions for which 50% tumor volume reduction is observed, a is a volume variation constant per number of breaks, and β is a parameter in Gy⁻² of a linear quadratic model relative to the tumor tissue type.

33. A method for evaluating a tumor response to a treatment, the method comprising:
preparing a cell sample from cells taken from said tumor;
subjecting said cell sample to a DNA-breaking treatment at a dose D;
determining a mean number N_{pH2AX}(t) of nuclear foci obtained with a marker pH2AX at observation times on said cell sample, said observation times being time t=0 min (called t0, state before administration of said dose D) and at least one predetermined observation time after administration of said absorbed dose D, the at least one predetermined observation time including a first predetermined observation time, a second predetermined observation time, a third predetermined observation time, and a fourth predetermined observation time; and
determining, using at least the mean numbers N_{pH2AX}(t), at least one parameter or score which makes it possible to characterize a response of the sample to said DNA-breaking treatment,
wherein:
the fourth predetermined observation time is a time for which a level of DNA breaks reaches its residual value;
the third predetermined observation time is a time after which approximately 25% of double-strand breaks (DSBs) are repaired in control cells from radioresistant patients;
the second predetermined observation time is a time after which approximately 50% of the DSBs are repaired in control cells from radioresistant patients; and
the first predetermined observation time is a time after which a number of recognized DSBs reaches a maximum in control cells from radioresistant patients.

34. The method of claim 33, wherein:
the fourth predetermined observation time is between 6×t3 and 8×t3;
the third predetermined observation time is between 3×t2 and 5×t2;
the second predetermined observation time is between 5×t1 and 7×t1; and
the first predetermined observation time is 10 minutes after discontinuing radiation.

35. The method of claim 33, wherein:
the fourth predetermined observation time is approximately 24 hours;
the third predetermined observation time is approximately 4 hours;
the second predetermined observation time is approximately 60 minutes; and
the first predetermined observation time is 10 minutes after discontinuing radiation.
